(19) United States
(12) Patent Application Publication
(10) Pub. No.: US 2012/0245661 A1
(43) Pub. Date: Sep. 27, 2012

(54) Cold Therapy Safety Switch

(76) Inventor: Jeffrey T. Mason, Escondido, CA (US)

(21) Appl. No.: 13/070,713

(22) Filed: Mar. 24, 2011

(51) Int. Cl. A61F 7/10

(52) U.S. Cl. 607/104

(57) ABSTRACT

The present invention relates to a temperature probe measuring the skin temperature of a patient at an injury or post-surgery site, a cold therapy unit cooling the patient at the site to reduce pain and swelling, and a safety shut off switch for powering off the cold therapy unit when the skin temperature drops below a threshold temperature. When the skin temperature drops below the threshold temperature, the cold therapy unit is shut off by opening the switch. When the skin temperature rises to or above the threshold temperature, the cold therapy unit is powered on again.

### BACKGROUND OF THE INVENTION

1. Field of Invention

The present invention relates generally to cold compression therapy, and specifically to improving the safety of a cold therapy unit which reduces pain and swelling at an injury site of a patient.

2. Description of Related Art

Cold therapy also known as cryotherapy (e.g., ice) reduces pain and swelling, otherwise known as edema formation, from an acute injury or post-surgery site. A further benefit of cryotherapy is the reduction of blood flow and the slowing of local cell metabolism. Cold therapy is especially useful for injuries such as sprains, strains, pulled muscles, and pulled ligaments during sports and other activities. The therapy is also often recommended by orthopedic surgeons following surgery, as ice is one of the principles of Rest, Ice, Compression, and Elevation (RICE) therapy.

According to the medical community's guidelines, icing with ice or gel pads should generally be limited to a maximum of 15-20 minutes at a time and no more frequently then every two hours the first day a soft tissue injury occurs and no more than two to four times a day after that until pain and swelling diminishes. Exceeding these established limitations puts the patient at risk for further injury. For example, exceeding the 20 minute treatment limitation can cause skin, nerve, and tissue damage, scarring, and frost-bite so severe that amputation may be required. Many other injuries can be caused, such as reflex sympathetic dystrophy, which is also known as complex regional pain syndrome.

It was found that if the skin temperature could be kept above 45° F. then the cold therapy could be applied continuously without the risk of tissue damage. This would have the advantage of continuous pain relief and reduction of swelling. Cold therapy units were developed to meet this need. These units consist of a reservoir containing a cold treatment fluid (i.e., ice and water), a treatment pad, a feed line and return line connecting the treatment pad to the reservoir, a thermometer in the return line to monitor the temperature of the treatment pad and an electric pump to circulate the treatment fluid through the treatment pad. The temperature of the treatment pad is controlled by changing the rate of flow through the treatment pad. A reduced flow allows the treatment fluid to remain in the pad for a longer period allowing the body to warm the fluid. Conversely, increasing the flow rate reduces the amount of time that the body's heat has to raise the temperature of the treatment fluid.

These cold therapy units have the same risk of injury should the skin temperature fall too low. In fact, it could be said that they have a higher risk of injury since it is assumed that they can be run continuously while it is generally known that ice and gel pads should only be applied intermittently.

The cold therapy units monitor the temperature of the fluid in the treatment pad not the skin temperature, which are assumed to be close, but sometimes they are significantly different, especially when there are gauzes and bandages insulating the skin from the treatment pad. For example, if the patient is supposed to be cooled to 45° F., the operator (e.g., a nurse or patient) may see the water temperature at 35° F. indicating that the skin is too cold, when in reality the skin temperature may be 55° F., which is too warm. Too correct what is thought to be too cold, the operator would turn the temperature up higher, when in this instance, the operator should be turning the temperature down lower. A doctor or nurse might realize that layers of gauze and bandages will insulate the skin from the treatment pad and will therefore tell the patient that they can set the temperature lower than 45° F. to compensate. At this point they are just guessing at what the correct setting should be. If the bandages should then get wet from wound leakage, the isolation value would be greatly reduced which would result in the skin temperature being too cold.

Even when the skin temperature and pad temperature are virtually the same, there is still a risk of injury. There are times when the patient is not instructed in the proper use of the cold therapy unit and feels “if a little is good, a lot is better” or “no gain without pain.” Other patients may just assume that the cold therapy unit was adjusted correctly at the hospital and needs no further adjustment when they take it home. Then, there is the patient that has been running the unit all day and diligently adjusted the temperature as instructed. Towards the end of the day the ice would have almost all melted and therefore the water in the reservoir would have warmed up. This would have required the patient to increase the flow to maintain the desired temperature at the pad. Then, just before going to bed, he replenishes the ice in the reservoir and goes to sleep without monitoring the temperature and readjusting the flow rate. This could easily result in many hours of near freezing treatment. Mistakes may cause harm such as tissue or nerve damage, especially when a powerful machine such as the cold therapy unit is used. This is because the cold therapy unit stays cold longer than ice and gel packs.

Ironically, a machine which is designed to reduce pain and to help a patient recover from injuries, has become better known for increasing pain and injuries. Many lawsuits have been filed for injuries related to cold therapy units. Many injuries that are caused by applying too much cold for too long of a period have symptoms very similar to those seen on patients after surgery that had no cold therapy applied. For example, it is not uncommon for patients after surgery to have blistering of the skin caused by swelling and heat from increased blood flow around the surgery site. Similarly, applying too much cold can cause similar blistering. Between the injuries that are caused by these cold therapy units and the injuries that just appear to be caused by them, the manufacturers of these units have become a very popular target among attorneys.

Also, the existence of this confusion makes it difficult to determine who is at fault when an injury occurs. On one hand, a similar amount of patients are injured each year using old fashioned ice and gel packs, because they incorrectly believe that “the more, the better” and “no pain, no gain.” These types of injuries may become more frequent and exaggerated given the power of the cold therapy units. On the other hand, a patient's injury may come from turning the temperature gauge in the wrong direction by being confused by the water temperature reading. This has led to each manufacturer spending millions of dollars each year between litigation and injury compensation, because it cannot be proved whether or not the injuries were caused by user error or whether the injury was even caused by excessive cold.

U.S. Pat. No. 6,551,348 to Blalock is directed to a temperature controlled therapy device which maintains a desired temperature in a fluid. The temperature controlled therapy device includes a fluid reservoir, a temperature controlled fluid, a pump for circulating the temperature controlled fluid through a fluid circuit, a differential temperature sensor for generating an output signal proportional to a difference in fluid temperature, a control circuit having as inputs the outputs of the differential temperature sensor and the absolute temperature sensor for generating a control signal for operating the pump in order to maintain a defined temperature range, and a power supply for supplying power to the device. An alternative embodiment controls the fluid temperature based on the differential temperature between the skin temperature and the fluid temperature. However, the Blalock device requires slowing the water flow through the treatment pad to warm the water. The problem with using ice and water for the treatment fluid and then controlling the temperature by controlling the pump output, is that in order to get the temperature up to a safe temperature the flow needs to be so slow that the pad collapses and only a small stream of water trickles through the pad. This is especially true when a compression wrap is applied over the pad. To keep the pad inflated and at the same time reduce the flow so as to raise the water temperature to a safe level, it is necessary to restrict the return or outlet conduit from the pad to the reservoir. Because of U.S. Pat. No. 5,330,519, only one of the dozens of units manufactured has that restrictor. The rest including the Blalock device rely on high flow rate to keep the pad inflated. Another drawback to the Blalock device is that it requires a comparatively complicated and therefore expensive circuit board with a motor control circuit.

It would be highly desirable to have safety device that would monitor the skin temperature and temporarily stop the flow of treatment fluid in a cold therapy unit when the skin temperature dropped below a safe level. It would further be desirable if the safety device would work with most of the current cold therapy units with little or no modification to the units. It would also be desirable if the device would be easy to use and inexpensive to manufacture.

### SUMMARY OF THE INVENTION

Embodiments of the present invention include a temperature probe measuring the skin temperature of a patient at an injury or post-surgery site, a cold therapy unit cooling the patient at the site to reduce pain and swelling, and a safety shut off switch for powering off the cold therapy unit when the skin temperature drops below a threshold temperature.

In one embodiment of the invention, a system comprises: a safety switch having an electrical input and an electrical output, the electrical input of the safety switch configured to receive power from a power source configured to power a cold therapy unit, the electrical output connected to a power input jack of the cold therapy unit, the safety switch having an open position and a closed position, the cold therapy unit receiving power through the safety switch when the safety switch is in the closed position and blocking power to the cold therapy unit when the safety switch is in the open position; a treatment pad configured to be coupled to the cold therapy unit and applied to skin of a patient at a treatment site, the treatment pad configured to supply cold therapy to the treatment site to reduce pain and swelling at the treatment site; and a temperature probe configured to be coupled to the safety switch and applied to the skin at the treatment site, the temperature probe measuring skin temperature at the treatment site, the safety switch providing power to the cold therapy unit when the skin temperature is at or above the threshold temperature, the safety switch blocking power to the cold therapy unit when the skin temperature is below the threshold temperature.

The power source may be a voltage transformer comprising an input and an output, the input of the voltage transformer receiving power from a wall socket and the output of the voltage transformer providing power to the electrical input of the safety switch. The output of the voltage transformer may comprise 12 volts. The electrical output of the safety switch may comprise 12 volts when the safety switch is in the closed position and 0 volts when the safety switch is in the open position. The threshold temperature may comprise 45 degrees Fahrenheit, and the safety switch powers the cold therapy unit when the skin temperature is greater than or equal to 45 degrees Fahrenheit, and the safety switch blocks power from getting to the cold therapy unit when the skin temperature is less than 45 degrees Fahrenheit. The power source may be household current at a wall socket. The electrical output of the safety switch may comprise household current when the safety switch is in the closed position and 0 volts when the safety switch is in the open position. The temperature probe may comprise a temperature sensor.

In another embodiment of the invention, a method comprises the steps of: receiving power from a power source at a switch located before a cold therapy unit, the cold therapy unit configured to cool tissue of a patient at a treatment site; measuring the skin temperature of the patient at the treatment site; providing power through the switch when the skin temperature is at or above a threshold temperature; and blocking power at the switch when the skin temperature is below the threshold temperature. The power source may comprise a voltage transformer or household current. The method may further comprise a temperature sensor for measuring the skin temperature. The temperature probe may comprise a temperature sensor. The switch may comprise an emergency shut off switch.

In another embodiment of the invention, a system comprises: a safety switch having an electrical input and an electrical output, the electrical input of the safety switch configured to receive power from a power source, the electrical output connected to an electric pump of a cold therapy unit, the safety switch having an open position and a closed position, the electric pump receiving power through the safety switch when the safety switch is in the closed position and blocking power to the electric pump when the safety switch is in the open position; a treatment pad configured to be coupled to the cold therapy unit and applied to a patient at a treatment site, the treatment pad receiving a cooling fluid from the electric pump to reduce pain and swelling at the treatment site; a temperature probe configured to be coupled to the safety switch and applied to the skin at the treatment site, the temperature probe measuring skin temperature at the treatment site, the safety switch providing power to the pump when the skin temperature is at or above the threshold temperature, the safety switch blocking power to the pump when the skin temperature is below the threshold temperature. The power source may be a voltage transformer comprising an input and an output, the input of the voltage transformer receiving power from a wall socket and the output of the voltage transformer providing power to the electrical input of the safety switch. The output of the voltage transformer may comprise 12 volts. The electrical output of the safety switch may comprise 12 volts when the safety switch is in the closed position and 0 volts when the safety switch is in the open position. The threshold temperature may comprise 45 degrees Fahrenheit, the safety switch powers the cold therapy unit's pump when the skin temperature is greater than or equal to 45 degrees Fahrenheit, and the safety switch blocks power from getting to the pump when the skin temperature is less than 45 degrees Fahrenheit. The power source may be household current at a wall socket. The temperature probe may comprise a temperature sensor.

In a further embodiment, a device comprises: an electrical input configured to receive power from a power source, a switching component having an input and an output, the input of the switching component coupled to the electrical input; and an electrical output coupled to the switching component, the electrical output configured to be connected to a cold therapy unit, the switching component having an open position and a closed position, the electrical output receiving power through the switching component when the switching component is in the closed position and blocking power to the cold therapy unit when the switching component is in the open position.

### BRIEF DESCRIPTION OF THE DRAWINGS

FIG. 1 illustrates a system for safely cooling a patient according to one embodiment of the invention.

FIG. 2 illustrates the electronics within the housing according to one embodiment of the invention.

FIG. 3 illustrates a cold therapy unit of the system according to one embodiment of the invention.

FIG. 4 illustrates the cold therapy unit attaching to a patient according to one embodiment of the invention.

FIG. 5 illustrates a flow chart showing a process for safely cooling a patient according to an embodiment of the invention.

### DETAILED DESCRIPTION OF THE INVENTION

Before the present composition, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

The present invention relates to a temperature probe measuring the skin temperature of a patient at an injury or post-surgery site, a cold therapy unit cooling the patient at the site to reduce pain and swelling, and a safety shut off switch for powering off the cold therapy unit when the skin temperature drops below a threshold temperature. When the skin temperature drops below the threshold temperature, the cold therapy unit is shut off by opening the switch. When the skin temperature rises to or above the threshold temperature, the cold therapy unit is powered on again. Advantageously, embodiments of the present invention reduce patient injuries and litigation related to cold therapy units.

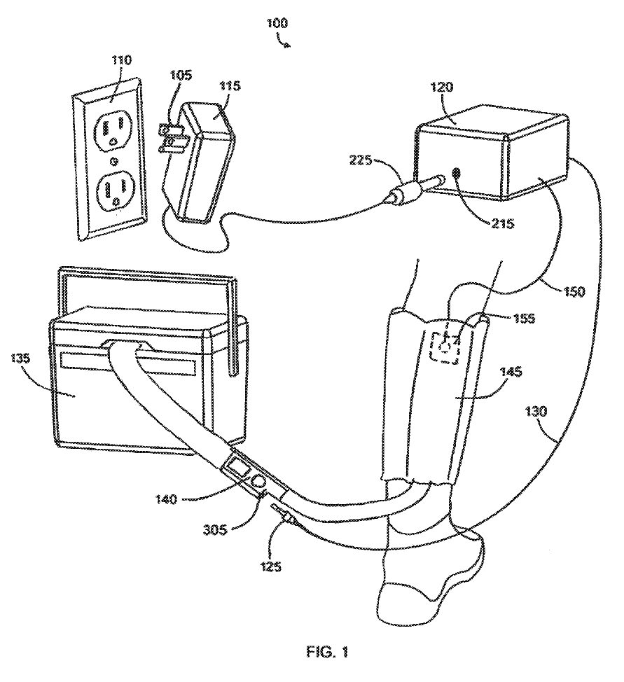

FIG. 1 illustrates a system 100 for safely cooling a patient according to one embodiment of the invention. The system 100 includes a wall plug 105 configured to receive power from a wall socket 110, a voltage transformer 115, a plug 225 on the output side of transformer 115, a safety switch 120 having an input power port 215 configured to receive the power plug 225, a power cord 130 leading to a power plug 125, a cold therapy unit 135 having a treatment pad 145, a temperature controller 140 with a power input port 305 configured to receive the power plug 125, and a temperature probe 150 having a temperature sensor 155.

The cold therapy unit 135 was originally configured to receive power directly from the power plug 225 to the power input port 305. The system 100 includes the safety switch 120 located between the power plug 225 and the input power port 305. This is a safer way to cool a patient using the cold therapy unit 135. The wall plug 105 can receive power from the wall socket 110 or another power source. The wall plug 105 transfers power to the voltage transformer 115. The voltage transformer 115 (e.g., a 12 volt wall transformer) provides power to the safety switch 120 by way of the power plug 225. The safety switch 120 receives the skin temperature from the temperature probe 150. If the skin temperature is above a predetermined threshold, the safety switch 120 will pass power through the power cord 130 and the power plug 125 and to the power input port 305. If the skin temperature falls below a predetermined threshold, the safety switch 120 will block power to power cord 130 therefore blocking all power to the cold therapy unit 135. In another embodiment, the power plug 225 is designed to not fit into power the input port 305 of the cold therapy unit 135 therefore precluding the use of the cold therapy unit 135 without the safety switch 120, preventing user error injuries that the safety switch 120 can prevent. In a further embodiment, the safety switch 120 is positioned between the wall plug 105 and the voltage transformer 115 such that the power is only supplied to the voltage transformer 115 when the safety switch is in the closed position.

The cold therapy unit 135 (e.g., cold therapy machine, cold therapy device, cold therapy system, etc.) has a temperature controller 140 with a knob for controlling the flow rate of the fluid, where the flow rate changes the temperature of the fluid. When the flow of water is slowed down, the body has time to heat the water, whereas when the flow of water is increased, the body has less time to heat the water, therefore the water stays colder.

An electric pump incorporated within cold therapy unit 135 is used to pump fluid into the treatment pad 145 via a hose, for cooling a patient. The temperature sensor 155 (e.g., thermocouple) is for measuring the skin temperature of the patient. The temperature sensor 155 can be located below the treatment pad 145 (e.g., within a sterile adhesive insulating bandage with typical dressings under the treatment pad 145) and isolated by insulation.

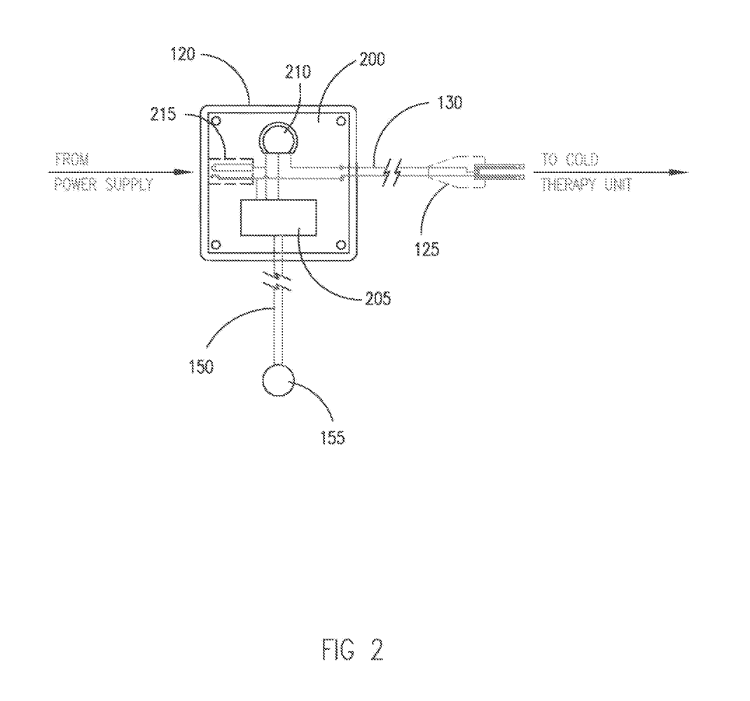

FIG. 2 illustrates electronics 200 within the safety switch 120 according to one embodiment of the invention. The electronics 200 (e.g., a control circuit) includes a processor 205, a switching component 210, a power input jack 215 (e.g., an electrical input), a power output cord 130 (e.g., an electrical output), and a temperature probe 150. The processor 205 receives temperature readings from the temperature sensor 155 and opens and closes the switching component 210 based on the input from the temperature sensor 155. The switching component 210 (e.g., shut off switch, emergency shut off switch, emergency switch, switch, etc.) passes power (e.g., 12 volts) to the cold therapy unit 135 through the power cord 130 when the switch is closed, and switching component 210 blocks power from reaching the cold therapy unit 135 when the switch is open. The switching component 210 opens when the measured skin temperature is below a threshold temperature to shut off the cold therapy unit 135. The switching component 210 closes again when the measured skin temperature rises to or above the threshold temperature.

The threshold temperature may be approximately 45° F. in one embodiment. In this embodiment, when the temperature is at or warmer than 45° F., the cold therapy unit 135 receives power through the switching component 210, and when the temperature drops below 45° F., the switching component 210 shuts off power to the cold therapy unit 135 until the temperature rises back to or above 45° F. In another embodiment, the threshold temperature is a temperature other than 45° F. (e.g., 30° F., 35° F., 40° F., 50° F., 55° F., 60° F., etc.)

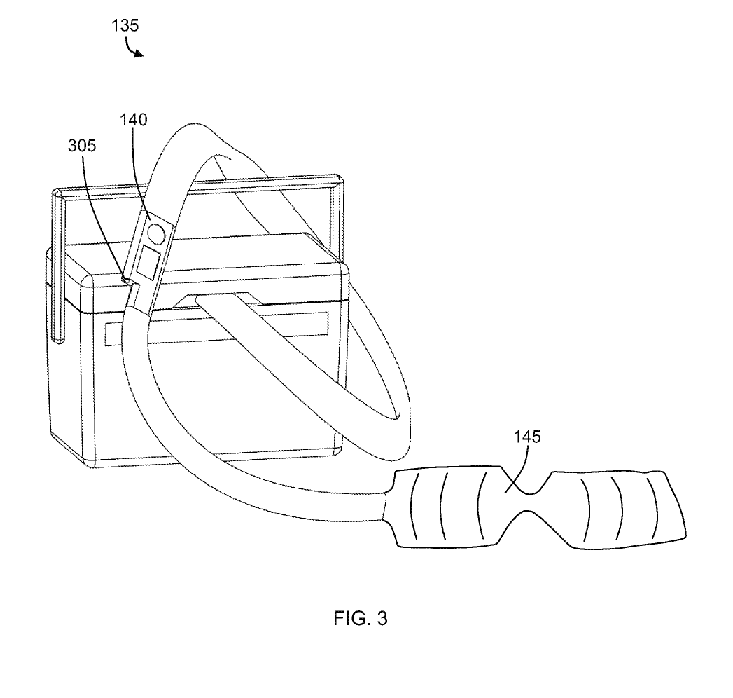

FIG. 3 illustrates the cold therapy unit 135 of the system 100 according to one embodiment of the invention. The cold therapy unit 135 includes a temperature controller 140 and a treatment pad 145. The temperature controller 140 includes a power input port 305 for receiving power from the power plug 125 of the safety switch 120. The temperature controller 140 controls the fluid temperature by controlling the flow rate, where a faster flow rate produces a colder temperature. The treatment pad 145 is configured to be applied to a treatment site on a patient.

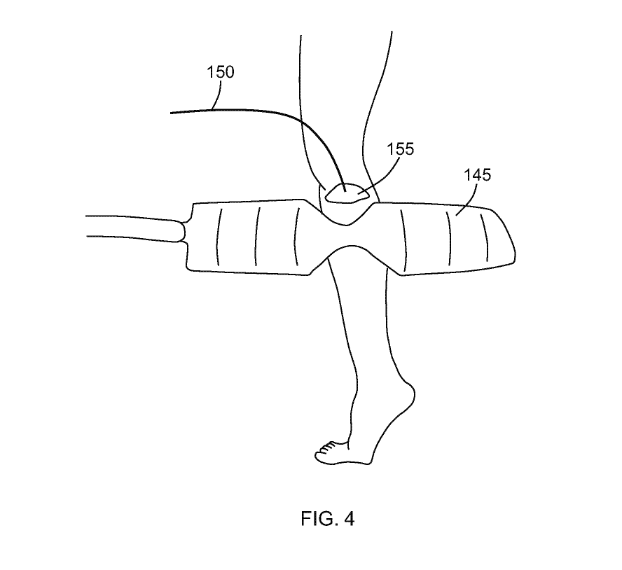

FIG. 4 illustrates the temperature sensor 155 applied to the patients skin prior to the treatment pad 145 of the cold therapy unit 135 being applied to the patient directly over the temperature sensor 155. The temperature probe 150 sends the temperature measurement to the processor 205.

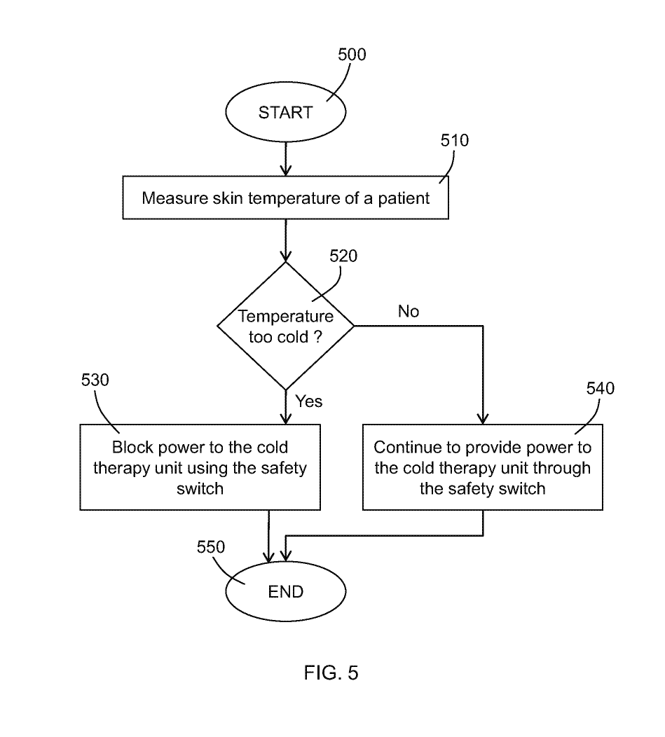

FIG. 5 illustrates a flow chart showing a process for safely cooling a patient according to an embodiment of the invention. The process starts at step 500. At step 510, the temperature sensor 155 measures the skin temperature of a patient. Then at decision step 520, the processor 205 determines if the temperature is too cold. If yes, the safety switch 120 blocks power from reaching the cold therapy unit 135 by the processor 205 opening the switching component 210 at step 530. Going back to decision step 520, if the temperature is not too cold, the safety switch 210 continues to provide power to the cold therapy unit 135 at step 540. The process ends at step 550.

It is to be recognized that depending on the embodiment, certain acts or events of any of the methods described herein can be performed in a different sequence, may be added, merged, or left out altogether (for example, not all described acts or events are necessary for the practice of the method). Moreover, in certain embodiments, acts or events may be performed concurrently, for example, through multi-threaded processing, interrupt processing, or multiple processors, rather than sequentially.

The steps of a method or algorithm described in connection with the embodiments disclosed herein may be embodied directly in a computer or electronic storage, in hardware, in a software module executed by a processor, or in a combination thereof. A software module may reside in a computer storage such as in RAM memory, flash memory, ROM memory, EPROM memory, EEPROM memory, registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC.

It should be understood that the invention is not limited to the embodiments set forth herein for purposes of exemplification, but is to be defined only by a fair reading of the appended claims, including the full range of equivalency to which each element thereof is entitled. Although the invention has been described with reference to the above examples, it will be understood that modifications and variations are encompassed within the spirit and scope of the invention. Accordingly, the invention is limited only by the following claims.

I claim herein:

1. A system comprising:
a safety switch having an electrical input and an electrical output, the electrical input of the safety switch configured to receive power from a power source configured to power a cold therapy unit, the electrical output connected to a power input jack of the cold therapy unit, the safety switch having an open position and a closed position, the cold therapy unit receiving power through the safety switch when the safety switch is in the closed position and blocking power to the cold therapy unit when the safety switch is in the open position;
a treatment pad configured to be coupled to the cold therapy unit and applied to skin of a patient at a treatment site, the treatment pad configured to supply cold therapy to the treatment site to reduce pain and swelling at the treatment site; and
a temperature probe configured to be coupled to the safety switch and applied to the skin at the treatment site, the temperature probe measuring skin temperature at the treatment site, the safety switch providing power to the cold therapy unit when the skin temperature is at or above the threshold temperature, the safety switch blocking power to the cold therapy unit when the skin temperature is below the threshold temperature.

2. The system of claim 1, wherein the power source is a voltage transformer.

3. The system of claim 2, wherein the voltage transformer comprises an input and an output, the input of the voltage transformer receiving power from a wall socket and the output of the voltage transformer providing power to the electrical input of the safety switch.

4. The system of claim 3, wherein the output of the voltage transformer comprises 12 volts.

5. The system of claim 1, wherein the electrical output of the safety switch comprises 12 volts when the safety switch is in the closed position.

6. The system of claim 1, wherein the output of the safety switch comprises 0 volts when the safety switch is in the open position.

7. The system of claim 1, wherein the threshold temperature comprises 45 degrees Fahrenheit.

8. The system of claim 7, wherein the safety switch powers the cold therapy unit when the skin temperature is greater than or equal to 45 degrees Fahrenheit.

9. The system of claim 7, wherein the safety switch blocks power from getting to the cold therapy unit when the skin temperature is less than 45 degrees Fahrenheit.

10. The system of claim 1, wherein the power source is household current at a wall socket.

11. The system of claim 10, wherein the electrical output of the safety switch comprises household current when the safety switch is in the closed position.

12. The system of claim 10, wherein the electrical output of the safety switch comprises 0 volts when the safety switch is in the open position.

13. The system of claim 1, wherein the temperature probe comprises a temperature sensor.

14. A method comprising:
receiving power from a power source at a switch located before a cold therapy unit, the cold therapy unit configured to cool tissue of a patient at a treatment site;
measuring the skin temperature of the patient at the treatment site;
providing power through the switch when the skin temperature is at or above a threshold temperature; and
blocking power at the switch when the skin temperature is below the threshold temperature.

15. The method of claim 14, wherein the power source comprises a voltage transformer.

16. The method of claim 14, wherein the power source comprises household current.

17. The method of claim 14, further comprising a temperature sensor for measuring the skin temperature.

18. The method of claim 14, wherein the switch comprises an emergency shut off switch.

19. A system comprising:
a safety switch having an electrical input and an electrical output, the electrical input of the safety switch configured to receive power from a power source, the electrical output connected to an electric pump of a cold therapy unit, the safety switch having an open position and a closed position, the electric pump receiving power through the safety switch when the safety switch is in the closed position and blocking power to the electric pump when the safety switch is in the open position;
a treatment pad configured to be coupled to the cold therapy unit and applied to a patient at a treatment site, the treatment pad receiving a cooling fluid from the electric pump to reduce pain and swelling at the treatment site; and
a temperature probe configured to be coupled to the safety switch and applied to the skin at the treatment site, the temperature probe measuring skin temperature at the treatment site, the safety switch providing power to the pump when the skin temperature is at or above the threshold temperature, the safety switch blocking power to the pump when the skin temperature is below the threshold temperature.

20. The system of claim 19, wherein the power source comprises a voltage transformer.

21. The system of claim 20, wherein the voltage transformer comprises an input and an output, the input of the voltage transformer receiving power from a wall socket and the output of the voltage transformer providing power to the electrical input of the safety switch.

22. The system of claim 21, wherein the output of the voltage transformer comprises 12 volts.

23. The system of claim 19, wherein the electrical output of the safety switch comprises 12 volts when the safety switch is in the closed position.

24. The system of claim 19, wherein the output of the safety switch comprises 0 volts when the safety switch is in the open position.

25. The system of claim 19, wherein the threshold temperature comprises 45 degrees Fahrenheit.

26. The system of claim 25, wherein the safety switch powers the cold therapy unit when the skin temperature is greater than or equal to 45 degrees Fahrenheit.

27. The system of claim 25, wherein the safety switch blocks power from getting to the cold therapy unit when the skin temperature is less than 45 degrees Fahrenheit.

28. The system of claim 19, wherein the power source comprises household current from a wall socket.

29. The system of claim 19, wherein the temperature probe comprises a temperature sensor.

30. A device comprising:
an electrical input configured to receive power from a power source,
a switching component having an input and an output, the input of the switching component coupled to the electrical input; and
an electrical output coupled to the switching component, the electrical output configured to be connected to a cold therapy unit, the switching component having an open position and a closed position, the electrical output receiving power through the switching component when the switching component is in the closed position and blocking power to the cold therapy unit when the switching component is in the open position.
